(19) United States
(12) Patent Application Publication
(10) Pub. No.: US 2010/0317903 A1
(43) Pub. Date: Dec. 16, 2010

(54) Integrated Process for Producing Diesel Fuel from Biological Material and Products, Uses and Equipment Relating to Said Process

(75) Inventor: Pekka Knuuttila, Porvoo (FI)

Correspondence Address: STEPTOE & JOHNSON LLP, 1330 CONNECTICUT AVENUE, N.W., WASHINGTON, DC, 20036, US

(73) Assignee: Upm-Kymmene OYJ, Helsinki (FI)

(21) Appl. No.: 12/742,209

(22) PCT Filed: Oct. 31, 2008
(86) PCT No.: PCT/EP2008/064823

§ 371 Date: Aug. 25, 2010

(30) Foreign Application Priority Data
Nov. 9, 2007 (FI) 20075794

(51) Int. Cl. C10L 1/16; C07C 1/20; C07C 7/04; B01J 8/00

(52) U.S. Cl. 585/14; 585/240; 585/800; 422/610

(57) ABSTRACT

The present invention relates to an integrated process for producing diesel fuel or fuel additive from biological material by producing paraffins by a Fischer-Tropsch reaction on one hand and by a catalytic hydrodeoxygenation of bio oils and fats on the other hand. The two hydrocarbon streams are combined and distilled together. The invention also relates to the use of lignocellulosic material, such as by-products of the wood-processing industry for producing diesel fuel and to a method for narrowing the chain length distribution of Fischer-Tropsch derived diesel fuel. The invention provides a high-quality middle distillate fraction from various biological sources and most preferably from by-products of the wood-processing industry. The invention also relates to equipment for producing fuel form biological material, which comprises a hydrodeoxygenation reactor (3) for hydrocarbons, a cracking/isomerization reactor (11) for FT paraffins and a separation unit (12) for the combined hydrocarbons. Hydrogen is separated from light fractions in a separation unit (9) and reformed in the process. The equipment is advantageously integrated with a pulp and paper mill, which provides biological material and receives waste and energy.

### FIELD OF THE INVENTION

The present invention relates an integrated process for producing diesel fuel from biological material by producing paraffins by a Fischer-Tropsch (FT) reaction on one hand and by a catalytic hydrodeoxygenation (HDO) of biological hydrocarbons on the other hand. The invention also relates to the use of lignocellulosic material, such as by-products of the wood-processing industry for producing diesel fuel and to a method for narrowing the chain length distribution of Fischer-Tropsch derived diesel fuel. The invention provides a high-quality middle distillate fraction from various biological sources and most preferably from by-products of the wood-processing industry. The invention also provides use of said fraction as a cetane improving additive. Furthermore, the invention relates to equipment for producing fuel form biological material.

### BACKGROUND OF THE INVENTION

The diminishing reserves of fossil fuels and the emission of harmful gases connected with their use have increased the interest in utilizing biological materials, especially from non-edible renewable resources for making liquid fuels capable of replacing fossil ones. Several prior art processes are known for producing liquid fuels from biological starting materials. One that has reached commercial success comprises the production of biodiesel (FAME) by transesterification of biomass-derived oils with alcohols. Due to the high pour point and unstability of FAME as well as its incompatibility with cars utilizing particle filters the use of this method is however restricted.

Biomass gasification processes have been in use for years for converting biomass into energy sources. Among the oldest applications is gasification and use of the producer gas mixture (CO+H₂) directly as a fuel in internal combustion engines. Almost any kind of biomass with a moisture content of 5% to 35% can be gasified. Examples of suitable biomass sources include forest slash, urban wood waste, by-products and waste of the papermaking industry, lumber waste, wood chips, sawdust, straw, firewood, agricultural residue, dung and the like.

Under controlled conditions, characterized by low oxygen supply and high temperatures, most biomass materials can be converted into a gaseous fuel known as synthesis gas. U.S. Pat. No. 4,592,762 describes gasification of cellulosic biomass in a single vessel wherein the cellulosic biomass is introduced directly into a single back-mixed fluidized bed of high heat capacity inert solids. U.S. Pat. No. 4,968,325 discloses a gasification plant having a pressure vessel containing a hot fluidized sand bed. The bio-mass is pre-dried to a moisture content of from 10% to 35% by weight. The fluidized bed is held at an operating temperature of 750 to 860° C. under an operating pressure of 400 kPa to 1750 kPa by controlling the feeding rate of the fluidized gas as well as the feeding rate of the biomass.

Synthesis gas from both renewable and fossil sources has been used to produce liquid fuels by the Fischer-Tropsch synthesis. US 2007/0225383 discloses a process for converting biomass to synthesis gas and providing a Fischer-Tropsch reaction for reacting the gas into fuels and chemicals. The invention improves the energy balance of the reaction by utilizing the heat of the exothermic Fischer-Tropsch reaction in the endothermic gasification reaction.

A standard three-phase slurry Fischer-Tropsch process typically employs heterogeneous cobalt catalysts and yields essentially n-paraffin hydrocarbons with a wide molecular weight distribution, depending on the set of operating conditions. The obtained mixture of hydrocarbons needs to be processed in subsequent process units to be valid as fuels. A typical processing includes an isomerization/cracking step using known catalysts containing molecular sieves or zeolites and group VII metals on a carrier.

The catalyst for the Fischer-Tropsch paraffins should have both cracking and isomerization functions. The cracking cuts up the long hydrocarbons to shorter (middle distillate) diesel range chains and the isomerization adds methyl side groups along the carbon chain thus lowering the pour and cloud points of the middle distillate products. Intermediate pore size aluminosilicate zeolites (ZSM) and silicoaluminophosphate (SAPO) molecular sieves have been proposed as catalyst for the dewaxing process.

It is well known in the art that intermediate pore silicoaluminophosphate molecular sieve SAPO's isomerize long chain alkanes rather than cracking them while the intermediate pore size aluminosilicate zeolites ZSM-5 and ZMS 23 perform both cracking and isomerization. Thus, U.S. Pat. No. 5,833,837 discloses a process wherein a waxy petroleum feed stock is distilled and two separate wax fractions, a heavy and a light lube base oil, are obtained. The light fraction is isomerized by a non-cracking SAPO type catalyst, while the heavy fraction is treated by a cracking ZSM-5 zeolite.

It is also known to produce liquid fuels from biological raw materials containing glycerol esters of fatty acids or free fatty acids. These raw materials contain high amounts of oxygen and minor amounts of sulphur, phosphorous and nitrogen, which are know to be catalyst poisons. U.S. Pat. No. 4,992,605 discloses the production of C₁₅ to C₁₈ paraffins by hydroprocessing vegetable oils such as canola, sunflower, soybean or rapeseed oils. U.S. Pat. No. 5,705,722 discloses a process for producing diesel range hydrocarbon components by hydrodeoxygenating (HDO) vegetable oils, tall oil, fractionated tall oil, animal fats and mixtures thereof. The feedstock is contacted with gaseous hydrogen and a hydrodesulfurization catalyst such as NiMo/Al₂O₃ or CoMo/Al₂O₃ in hydroprocessing conditions. The resulting product is described as an additive for diesel fuels having a high cetane number.

FI 100248 discloses a two-step process for producing middle distillate from vegetable oil by first hydrogenating the oils to n-paraffins and then by isomerizing the paraffins to obtain branched paraffins. U.S. Pat. No. 7,232,935 proposes an improvement of FI 100248 by suggesting the use of a pre-hydrogenation step prior to the hydrodeoxygenation and by operating the isomerization in a counter-current manner. U.S. Pat. No. 7,279,018 discloses a fuel composition which comprises a) a component derived from animal fats by hydrogenation and isomerization and b) a component containing oxygen, said components a) and b) being mixed or dissolved in c) a diesel component based on crude oil or a Fischer-Tropsch fraction. US 2007/0068848 discloses a process for providing a diesel fuel of high cetane number from triglycerides by a combination of thermal cracking and catalytic hydrotreating followed by distillation to recover a diesel fuel fraction with a cetane value of 70 to 80.

Despite the ongoing research and development in the use of renewable resources for producing fuel, there is still a need to improve the processes and to provide high quality diesel fuel from low grade materials and with little or no utilization of edible plant resources. There is also a need to use industrial waste materials close to the site of production in order to avoid high costs of transportation and storage.

The present invention provides a high grade diesel fuel from totally biological resources by the industrial integration of two separate processes for producing biological fuel.

### SUMMARY OF THE INVENTION

The present invention relates to an integrated process for producing diesel fuel from biological material. The process is characterized by the steps of a) providing a first hydrocarbon stream comprising C₁ to C₁₀₀₊ hydrocarbons by catalytic cracking/isomerization of Fischer-Tropsch paraffins of biological origin, b) providing a second hydrocarbon stream comprising predominantly C₁₅ to C₁₈ hydrocarbons by catalytic hydrodeoxygenation of biological hydrocarbons, c) mixing said first and second hydrocarbon streams, d) fractionating the resulting mixed hydrocarbon stream, and e) recovering a middle distillate fraction, preferably enriched in C₁₅ to C₁₈ hydrocarbons.

An object of the present invention is to combine hydrodeoxygenation (HDO) and Fischer-Tropsch (FT) processes to produce high quality paraffinic biological diesel fuel, free from aromatics, oxygen and sulphur. An object is to produce a fuel which is superior in quality compared to both crude fossil based diesel fuel and FAME biodiesels.

Another object of the invention is to maximize the utilization of process unit operations, which are common to both FT and HDO fuel production processes.

According to an embodiment of the integrated process of the present invention hydrogen is recirculated throughout the process. Hydrogen is both produced and consumed in the various reactions taking place in the integrated process. By providing a combined recovery, reforming and recirculation unit for hydrogen, technical benefits are gained in the process.

An advantage of the process integration is the ability for an autonomous hydrogen production. This adds a degree of freedom in site location of the fuel production, as proximity to a petrochemical factory producing hydrogen is not needed. When wood-derived waste and by-products are used, the proximity to a pulp and paper mill provides key benefits for energy integration, feed logistics, storage and/or waste treatment.

The invention also relates to the use of lignocellulosic material, such as waste and/or by-products of the wood-processing industry for producing diesel fuel from purely biorenewable sources. The use according to the invention comprises the steps wherein, a) biomass comprising lignocellulosic material, such as wood-derived waste material and/or by-products are gasified and used to provide a Fischer-Tropsch paraffin stream, which is then cracked under isomerizing conditions, b) biological hydrocarbons, such as tall oil or tall oil fatty acid is hydrodeoxygenated to provide a predominantly C₁₅ to C₁₈ paraffin stream, preferably a n-paraffin stream, which is optionally isomerized under non-cracking conditions, c) the two streams are combined and fractionated, and d) a middle distillate fraction, preferably enriched in C₁₅ to C₁₈ hydrocarbons is recovered.

The present invention also relates to a method for narrowing the chain length distribution of a Fischer-Tropsch derived diesel fuel. The method comprises the steps of a) combining 5% to 95% of a Fischer-Tropsch derived hydrocarbon stream of C₅ to C₁₀₀₊ hydrocarbons with 5% to 95% of a predominantly C₁₅ to C₁₈ hydrocarbon stream obtained by hydrodeoxygention of biological hydrocarbons, b) fractionating the combined hydrocarbon stream, and c) recovering a fraction of C₁₁ to C₂₀ hydrocarbons, preferably enriched in C₁₅ to C₁₈ hydrocarbons.

The present invention further relates to a biological middle distillate fraction obtainable by the process according to the invention and comprising from 5% to 95% of a first Fischer-Tropsch derived hydrocarbon stream having carbon atoms between 11 and 20 and from 5 to 95% of a second hydrocarbon stream having predominantly 15 to 18 carbon atoms. The fraction typically contains at least 25% and preferably about 40% to 80% of C₁₅ to C₁₈ hydrocarbons. The fraction may contain even more than 80% C₁₅ to C₁₈ hydrocarbons.

The middle distillate fraction is useful as a diesel fuel as such. However, the invention also relates to the use of the biological middle distillate fraction as an additive for improving the cetane value and/or the cloud point or pour point of a fuel produced by other means.

The invention also provides equipment for producing fuel from biological material. The equipment comprises a hydrodeoxygenation reactor for hydrodeoxygenating a feed of biological hydrocarbons; a cracking/isomerization reactor for catalytically cracking and isomerizing a feed of Fischer-Tropsch paraffins of biological origin; a separation unit for distilling a combined feed of hydrocarbons provided from said reactors and for the recovery of a middle distillate boiling at 150 to 400° C. and the separation of a top fraction boiling at a lower temperature; a hydrogen separation unit, for example a hydrogen permeable membrane, for separation of hydrogen from said top fraction and means for feeding said hydrogen to said reactors, and means for providing of additional hydrogen for said reactors from the non-permeable portion of said top fraction.

In one embodiment of the invention, the integrated fuel producing equipment is further integrated with the equipment of a pulp and paper mill so that waste and by-products produced in the pulp and paper process are fed into the fuel process and so that energy and waste produced in the fuel process are supplied to the pulp and paper mill.

### BRIEF DESCRIPTION OF THE DRAWINGS

FIG. 1 is a schematic flow diagram representing an embodiment of the invention.

FIG. 2 is the diagram of FIG. 1 provided with mass balance measurement points.

### DETAILED DESCRIPTION OF THE INVENTION

The present invention provides an integrated process for producing high grade diesel fuel form two different sources of biological material. By combining the hydrocarbon streams from two different sources, an improved chain length distribution is obtained in the fractionation of the combined streams. A fuel with improved cetane value and cloud point is obtained by the process. Integration of the processes also provides technical advantages by the combination of distillation units, hydrogen recirculation and reforming, waste handling, energy production etc.

In the present specification and claims, the following terms have the meanings defined below.

An “integrated process” means a process wherein two or more related functions, which can be separately performed, are combined so that at least one significant process step is common for the two processes.

The term “biodiesel” refers in this specification only to those traditional biological fuel products which are produced from transesterification of biomass-derived oils with alcohol and which contain oxygen.

The “biological diesel fuel” or “diesel fuel of biological origin” which is produced according to the present invention is a diesel fuel based on other processes than transesterification. The diesel fuels of the present invention are practically free of oxygen.

The term “middle distillate” refers to a hydrocarbon fraction, wherein the hydrocarbons consist essentially of hydrocarbons typically having a carbon chain length of 11 to 20 (designated C₁₁ to C₂₀). The middle distillate fraction typically has a boiling point in the range of 150 to 400° C. and preferably 175 to 350° C. The middle distillate hydrocarbons are those typically used as diesel fuels. It should be noted that since distillation does not provide an absolute cut off at a specific chain length, the various distillate fractions may contain insignificant amounts of hydrocarbons having a slightly lower or slightly higher carbon chain lengths. The cut off point in the distillation varies slightly depending on the intended use and the desired properties of the middle distillate. Thus, a distillate fraction comprising a wider range of carbohydrates such as C₉ to C₂₂ or a narrower range of carbohydrates such as C₁₄ to C₁₈ should also be understood as a middle distillate fraction.

The term “heavy fraction” refers to a hydrocarbon fraction, wherein the hydrocarbons consist essentially of hydrocarbons having a carbon chain length above 20. This is designated in the specification as C₂₁ to C₁₀₀₊. The “100+” refers to an unspecific number of carbon atoms up to 100 and above, which depends on the conditions under which the FT process and cracking/isomerization is performed. Typically the amount of carbon chain lengths above 100 is small, but the fraction may even include molecules having a chain length of 200 or more.

The term “naphtha fraction” refers to a distilled hydrocarbon fraction, wherein the hydrocarbons consist essentially of hydrocarbons having a carbon chain length of 5 to 10 (designated C₅ to C₁₀). The naphtha fraction hydrocarbons are those typically used as light fuels, solvents or raw materials e.g. for further processes based on steam cracking.

The term “kerosene fraction” refers to a distillate hydrocarbon fraction included in the above defined middle distillate, wherein the hydrocarbons consist essentially of hydrocarbons having a carbon chain length of 11 to 15 (designated C₁₁ to C₁₅). The kerosene fraction hydrocarbons are those typically used as fuel in jet engines.

The term “light fraction” refers to a hydrocarbon fraction, wherein the hydrocarbon chain length is 1 to 4 (designated C₁ to C₄). The light fraction also includes other gaseous components such as hydrogen and carbon monoxide, depending on the process from which the light fraction derives.

The term “predominantly C₁₅ to C₁₈ hydrocarbons” refers to a stream wherein more than 60%, preferably more than 80% and most preferably more than 90% of the hydrocarbons in the stream contain 15 to 18 carbon atoms. Hydrocarbons from plant sources typically contain almost only C₁₄ to C₁₈ hydrocarbons with C₁₅ to C₁₈ being most abundant.

The term “synthesis gas” or “syngas” refers to a gas mixture that contains varying amounts of carbon monoxide and hydrogen generated by the gasification of a carbon containing substance. Gasification of biological materials provides a ratio of hydrogen to carbon monoxide, which is about 2. The gas is suitable for providing hydrocarbons by the Fischer-Tropsch synthesis especially after some additional hydrogen has been added.

The “Fischer-Tropsch” synthesis is a catalyzed chemical reaction in which hydrogen and carbon monoxide are converted to a substantially Gaussian distribution of hydrocarbon chains of various lengths (designated (C₁ to C₁₀₀₊). Typical catalysts used are based on iron and cobalt. The term “Fischer-Tropsch conditions” refers to reaction conditions which are suitable for conducting a Fischer-Tropsch reaction. For producing diesel fuels the so called alfa-value (the alfa-value a value between 0 and 1 which is lowest for methane and highest for solid waxes) should be high and preferably close to 0.89, which is the maximum alfa-value for middle distillates. Such conditions are well known and documented in the art.

The term “separation” refers to the separation of hydrocarbons of different chain length of the FT paraffins of biological origin into different fractions alternatively within the FT process or by one or more separate process units. The separation is performed by distillation, flash separation where gas (vapor) is separated from liquid components under reduced pressure, by condensing or by any other suitable separation method.

The term “narrow chain length distribution” refers to a hydrocarbon stream or fraction having significantly more members of certain chain length(s) than what would be provided by a Gaussian distribution for said stream or fraction. A Fischer-Tropsch synthesis provides hydrocarbons with a broad (Gaussian) chain length distribution (C₁ to C₁₀₀₊), while hydrodeoxygenation of biological oils, fats, waxes or fatty acids provide a very narrow chain length distribution (predominantly C₁₅ to C₁₈).

The term “catalytic hydrodeoxygenation” (HDO) used in the present specification and claims refers to a catalytic treatment of the biological hydrocarbon feed, such as oil, fat, wax and/or fatty acid, with hydrogen under catalytic conditions, wherein the following reactions take place: breaking down of the structure of the hydrocarbons, deoxygenation or removal of oxygen as water, and hydrogenation to saturate double bonds. The preferred HDO of the invention also removes non-desired impurities such as sulphur as hydrogen sulfide and nitrogen as ammonia. Useful HDO catalysts are, for instance, those mentioned in U.S. Pat. No. 7,232,935 as suitable for the HDO step described therein.

The term “biological hydrocarbons” used in the present specification and claims refers to a feedstock comprising vegetable oils, animal fats, fish oils, natural waxes, fatty acids and mixtures thereof. Plant, animal and/or fish based biological triglyceride and/or fatty acid raw material in the form of oils, fats, waxes and/or acids are examples of the feed used for the HDO process of the present invention. Preferred feeds are crude tall oil, tall oil fatty acids and depitched tall oil.

The terms “isomerization” and “hydroisomerization” refer to the catalytic and hydrogen assisted introduction of short chain (typically methyl) branches into paraffinic hydrocarbons, preferably n-paraffinic hydrocarbons.

The term “non-cracking hydroisomerization”, which is used for the HDO hydrocarbon stream of the present invention, refers to an isomerization performed with a catalyst known to have little or no effect on cracking of the hydrocarbons in question. Typical non-cracking catalysts comprise intermediate pore size silicoaluminophosphate molecular sieve (SAPO) catalysts. Useful non-cracking isomerization catalysts are, for instance, those mentioned in U.S. Pat. No. 7,232,935 for the isomerization step described therein.

The term “catalytic cracking/isomerization”, which is used for the FT produced paraffins according to the present invention, refers to a simultaneous cracking and hydroisomerization step which is performed in the presence of hydrogen with a catalyst known to have both cracking and isomerizing properties. Typical cracking/isomerizing catalysts include ZSM zeolite catalyst such as the ZSM-5 and ZSM-23 catalysts described U.S. Pat. Nos. 4,222,855, 4,229,282 and 4,247,388 for use in selective cracking and isomerization of a paraffinic feedstock such as a Fischer-Tropsch wax.

The term “autothermal reforming” refers to the catalytic production of hydrogen from feed stocks such as hydrocarbons and methanol by the combination of partial oxidation and steam reforming.

The term “water gas shift” refers to the inorganic chemical reaction in which water and carbon monoxide react to form carbon dioxide and hydrogen (water splitting).

The terms “biological material” and “biological origin” refer to a wide variety of biomass derived from plants, animals and/or fish, i.e. from biorenewable sources as opposed to fossil sources.

The term “lignocellulosic material” refers to plant biomass that is composed of cellulose and hemicellulose, and lignin. Biomass comes in many different types, which may be grouped into four main categories: wood residues, including sawmill and paper mill discards, municipal paper waste, agricultural residues, including corn stover (stalks and straw) and sugarcane bagasse, and dedicated energy crops, which are mostly composed of tall, woody grasses.

The term “wood-processing industry” refers to any kind of industry that uses wood as its raw material. Typical wood-processing industries comprise pulp and paper mills, saw mills, panel board companies, fire-wood producers, wood pelletizers, etc.

The term “cetane number” or “cetane value” relates to the ignition quality of diesel fuel. It is a value obtained by a standardized comparison of the fuel under analysis with fuels or blends with a known cetane number. The reference fuel n-cetane (C₁₆) has the cetane number 100.

The term “cloud point” is a measure of the ability of a diesel fuel to operate under cold weather conditions. It is defined as the temperature at which wax first becomes visible when diesel fuel is cooled under standard test conditions.

The term “pour point” refers to the lowest temperature at which a diesel fuel flows when cooled under standard conditions.

The process of the present invention is operated in an integrated industrial plant with two main processes (FT and HDO) interconnected by a common fractionation process with recovery of a diesel fuel fraction as a product fraction and an optional recirculation of a light fraction for recovery and/or reforming of hydrogen.

The raw material for both of the main processes is of biological origin. For the FT process, almost any kind of biomass is suitable for being gasified. If needed, the biomass is first dried to bring its moisture content down to 35% or less. The biomass is typically selected from virgin and waste materials of plant, animal and/or fish origin, such as municipal waste, industrial waste or by-products, agricultural waste or by-products (including also dung), waste or by-products of the wood-processing industry, waste or by-products of the food industry, marine plants (such as algae) and combinations thereof. The biomass material is preferably selected from non-edible resources such as non-edible wastes and non-edible plant materials, including oils, fats and waxes. A preferred biomass material according to the present invention comprises waste and by products of the wood-processing industry such as slash, urban wood waste, lumber waste, wood chips, sawdust, straw, firewood, wood materials, paper, by-products of the papermaking or timber processes, etc. The biomass material for the FT process may also comprise vegetable oils, animal fats, fish oils, natural waxes, and fatty acids. Such oils, fats, waxes and acids are examples of the plant, animal and/or fish based biological raw material for the HDO process of the present invention. The raw material for the HDO process is preferably a non-edible oil such as jatropha oil, castor oil, tall oil fatty acids (TOFA) or tall oil, especially crude tall oil or depitched tall oil etc.

The raw materials useful in the process of the invention come in various different forms and they may be subjected to any suitable pre-handling process in order to improve their utility in the integrated fuel process of the invention. Thus, the material may be sorted, cleaned, washed, dried, ground, compacted, mixed, pre-hydrogenated, etc. in order to remove impurities and to provide a feed stream which is suitable for the synthesis gas production for the FT reaction and/or for the HDO reaction.

The first or FT hydrocarbon stream is typically provided by initial gasification of biomass feedstock to provide synthesis gas. The synthesis gas is then reacted in a Fischer-Tropsch (FT) reaction under conditions which provide C₁ to C₁₀₀₊ paraffins. After removal of off-gases, the remaining C₅ to C₁₀₀₊ paraffins of the paraffin stream are subjected to cracking/isomerization to shorten the chain length. This increases the proportion of C₁₁ to C₂₀ paraffins in the first hydrocarbon stream. At the same time, the isomerization provides branched hydrocarbons which improve the cloud point of the end product fuel.

The second or HDO hydrocarbon stream is typically provided by hydrodeoxygenation (HDO) of a biological hydrocarbon feedstock, for example a fat, oil, wax and/or fatty acid feedstock to provide a stream of saturated n-paraffins. Because the fatty acids of triglycerides of natural fats and oils typically have a chain length in which the hydrocarbons with 15 to 18 carbons are most abundant, the resulting paraffins will also contain predominantly C₁₅ to C₁₈ carbon chains. The HDO treatment saturates any unsaturated chains, wherefore the hydrocarbons will consist essentially of C₁₅ to C₁₈ n-paraffins. In one embodiment of the invention, the n-paraffins are hydroisomerized to increase the proportion of i-paraffins in the second hydrocarbon stream. However, the n-paraffins may be used as such to improve the cetane number of the FT hydrocarbon stream. The isomerization of the HDO hydrocarbons is performed with a non-cracking catalyst, to avoid shortening the C₁₅ to C₁₈ carbon chains. The hydroisomerization may be performed in a separate isomerization reactor or it may be performed in the same reactor with the hydrodeoxygenation.

The two hydrocarbon streams are then combined into a mixed hydrocarbon stream. In principle any mixing ratio is possible. However, for practical purposes and to provide the desired improvement in the end product, the mixed stream should contain 5% to 95% FT hydrocarbons and 5% to 95% HDO hydrocarbons. Preferably, the mixed stream contains 20% to 80% FT hydrocarbons and 20% to 80% HDO hydrocarbons. Most preferably, the amount of FT hydrocarbons is 40% to 60% and the amount of HDO hydrocarbons is 40% to 60%. A substantially 50/50 mixed stream has been found to be ideal because it provides a diesel fuel with a very narrow chain length distribution and improved properties both as regards the cetane number and cloud point. In a preferred embodiment the maximum cetane number is 100. The cloud point can be as low as −20° C.

The mixed hydrocarbon stream provided by combining the FT hydrocarbon stream with the HDO hydrocarbon stream is fractionated in a separator. The separator comprises a distilling unit in which the mixed feed is separated into different fractions based on selected boiling point ranges. The preferred fractionation provides a middle distillate containing C₁₁ to C₂₀ hydrocarbons and a light fraction containing hydrogen and C₁ to C₄ hydrocarbons. The middle distillate is recovered as an improved biological diesel fuel. It may also be used as an additive for blending with fuels provided by other means. It should be understood that the cut in the fractionation is not absolute and that some lighter and/or heavier hydrocarbons may be included in the separate fractions. Such lighter/heavier hydrocarbons are included in amounts which are insignificant for the intended use.

The combination of the two types of hydrocarbon streams and the distillation of the mixed stream improves the hydrocarbon distribution of the resulting middle distillate fuel since the HDO produced hydrocarbon stream is very rich in middle distillate (C₁₁ to C₂₀) hydrocarbons. Typically a HDO hydrocarbon stream from biological oils or fatty acids consists almost exclusively of C₁₄ to C₁₈ hydrocarbons, which are the most preferred ones in the middle distillate, since they improve the cetane value of the diesel fuel.

The resulting middle distillate fuel or fuel additive has an exceptionally good hydrocarbon distribution with more than 25% of the hydrocarbons being having 15 to 18 carbon atoms. In one embodiment the C₁₅ to C₁₈ hydrocarbon proportion is above 40% and it may be as high as 80% or even higher. This provides excellent ignition properties and a uniform combustion.

In one embodiment of the invention a naphtha fraction of C₅ to C₁₀ hydrocarbons and/or a heavy fraction of C₂₁ to C₁₀₀₊ is/are also recovered from the separator. The naphtha fraction is useful as such as a solvent. The heavy fraction may be recovered and used e.g. as a lubricating oil. In one embodiment of the invention, at least a part of the heavy fraction is recirculated to the FT cracking/isomerization step to be cracked and hydroisomerized for increasing the amount of C₁₁ to C₂₀ hydrocarbons in the first hydrocarbon stream. The heavy fraction may also be recirculated to the FT cracking/isomerization in total.

It is clear that other fractions than the above mentioned ones may also be recovered if so desired and that the mixed hydrocarbon stream or any of the above fractions may be distilled to provide other, slightly broader and/or narrower carbon chain length ranges.

When the distillation range is set to provide a fraction consisting essentially of C₁₅ to C₁₈ n- and i-paraffins, the resulting product is an ideal, high cetane/low cloud point fuel or fuel additive. In one preferred embodiment of the invention a kerosene fraction is also separated from the rest of the middle distillate.

By isomerizing one or both of the hydrocarbon feeds, the cloud point and the pour point of the resulting diesel fuel is significantly improved. Moreover, the biological HDO and FT processes both provide very clean hydrocarbon products essentially free of sulphur, phosphorus and nitrogen compounds. The resulting diesel fuel is also essentially free of aromatic hydrocarbons. Preferably the amount of aromatic hydrocarbons is below 1 w-%.

According to the preferred embodiment of the invention, a top fraction from the separation unit boiling at a temperature lower than the desired middle distillate is used in the integrated fuel process for providing hydrogen for the catalytic reaction(s) which result in the first and/or the second hydrocarbon stream(s). In one embodiment, the top fraction comprises the light fraction containing hydrogen and C₁ to C₄ hydrocarbons and it is directed to a hydrogen separation unit to separate hydrogen for recirculation to the cracking/isomerization step, to the hydrodeoxygenation step and/or to the hydroisomerization step.

The hydrogen separation unit typically comprises a hydrogen permeable membrane from which the hydrogen is recovered. In one embodiment of the invention the C₁ to C₄ hydrocarbons and any carbon monoxide recovered from the membrane and/or from other positions of the process are directed to a unit capable of converting the light feed stream into a gas comprising hydrogen, carbon monoxide, carbon dioxide, nitrogen and water. In one embodiment of the invention, the converting unit is an autothermal reforming (ATR) unit, which is known as such to those skilled in the art.

The gas produced in the ATR unit may be used for providing more hydrogen, for example by directing it to a water gas shift (WGS) unit to produce more hydrogen from carbon monoxide and water. The hydrogen is suitably separated out in a pressure swing adsorption (PSA) unit or the like. The hydrogen may also be separated in a hydrogen separation membrane, preferably the same membrane as the one used for the top fraction from the separator. The hydrogen is then recirculated in the process to the hydrodeoxygenation step, hydroisomerzation step and/or cracking/isomerization step.

In one embodiment of the invention, a light fraction of the Fischer-Tropsch paraffins containing hydrogen, carbon monoxide and C₁ to C₄ hydrocarbons, is also directed to the hydrogen separation unit. Furthermore, gases containing hydrogen and light fractions from the HDO stage and/or the isomerization stages may also be directed to the membrane to recover hydrogen and hydrogen-producing gases.

Hydrogen for the integrated process may be provided from any standard source such as methanol. However, by circulating and reforming hydrogen in an integrated manner, very little additional hydrogen will be needed in the integrated process. When additional hydrogen is required in the process it may be provided from any standard source. However, in one embodiment, the make-up hydrogen is produced by feeding methanol to the autothermal reforming unit. The methanol is preferably methanol produced from renewable sources, so called bio-methanol. The converting unit, i.e. for example the autothermal reforming unit, varies depending on the outside hydrogen source used.

In one embodiment the whole integrated fuel process of the present invention is operated in connection with a wood-processing industry and the biological feeds to the process originate in or as by-products of said wood-processing industry. In such a case, the biological origin of the FT hydrocarbon stream comprises waste or by-product(s) of the wood-processing industry and the biological hydrocarbons for the HDO hydrocarbon stream comprise tall oil or tall oil fatty acids. The wood-processing industry typically includes a Kraft mill producing tall oil or tall oil fatty acids or a mixture of tall oil fatty acids.

When the integrated fuel process is operated in connection with a wood-processing industry, the water produced in the integrated fuel process is typically supplied to said wood-processing industry for purification. Also sulphur freed in the integrated fuel process such as in the hydrodeoxygenation step and/or in the synthesis gas production is fed to the sulphur circulation of the wood-processing industry.

The greatest benefits of the present integrated fuel process are obtained by integrating it further with a pulp and paper mill. In such a case, lignocellulosic material, such as waste and/or by-products of the pulp and paper mill are very efficiently utilized as raw materials for the fuel production. At the same time, wastes of the fuel process such as water and hydrogen sulphide are efficiently handled in the traditional waste water treatment and sulphur recirculation systems, respectively, of the pulp and/or paper mill. Importantly also, energy released in the exothermic Fischer-Tropsch reaction of the fuel process is very useful for unit operations of the pulp and paper mill. The drying of the paper in the paper mill is thus advantageously performed with heat from the Fischer-Tropsch reaction.

In addition to the raw material, waste-handling and energy benefits of the doubly integrated fuel/wood-processing plant embodiment described above, there are benefits in the feed and storage logistics of the different streams passing between the fuel process and the wood-processing plant.

The present invention makes it possible to utilize lignocellulosic material, such as waste and/or by-products of the wood-processing industry for producing high quality diesel fuel from purely bio-renewable sources. In this case biomass comprising wood-derived waste material and/or by-products are gasified and used to provide a Fischer-Tropsch paraffin stream, which is then cracked under isomerizing conditions. Biological hydrocarbons, such as tall oil or tall oil fatty acid is hydrodeoxygenated to provide a predominantly C₁₅ to C₁₈ paraffin stream, preferably a n-paraffin stream, which is optionally isomerized under non-cracking conditions. The two streams are combined and fractionated, and a middle distillate fraction (C₁₁ to C₂₀) is recovered. Preferably a top fraction is used to provide hydrogen for recirculation to the production and/or isomerization of both hydrocarbon streams. The bottom fraction is preferably recirculated to cracking and isomerization.

The present invention also provides a method for narrowing of the chain length distribution of a Fischer-Tropsch derived diesel fuel. In the method, a a Fischer-Tropsch derived hydrocarbon stream of C₁ to C₁₀₀₊ hydrocarbons, from which the C₁ to C₄ hydrocarbons have been separated, is first combined with a predominantly C₁₅ to C₁₈ hydrocarbon stream obtained by hydrodeoxygenation of biological hydrocarbons. Then the combined hydrocarbon stream is fractionated, and a middle distillate fraction of C₁₁ to C₂₀ hydrocarbons enriched in C₁₅ to C₁₈ hydrocarbons is recovered. The recovered fraction typically contains at least 25% and preferably about 40% to 80% C₁₅ to C₁₈ hydrocarbons. For specific purposes, the fuel process may be operated so as to produce a fuel fraction containing more than 80% of C₁₅ to C₁₈. This may be achieved by selecting the biological hydrocarbon feed for the HDO, by optimizing the Fischer-Tropsch process conditions, by selecting a suitable ratio between the hydrocarbon streams and/or by adjusting the fractionation conditions and the boiling point range in the separator.

The equipment to be used in the construction of the integrated fuel process as well as in the doubly integrated fuel/wood-processing design typically comprises components which are known as such or may be modified from components known as such.

The basic equipment required for the integrated fuel process of the present invention includes a hydrodeoxygenation reactor for hydrodeoxygenating a feed of biological hydrocarbons, a cracking/isomerization reactor for catalytically cracking and isomerizing a feed of Fischer-Tropsch paraffins of biological origin, and a separation unit for distilling a combined feed of hydrocarbons provided from said reactors and for the recovery of a middle distillate boiling at 150 to 400° C.

By providing a hydrogen recovery unit, hydrogen recirculation lines and units for providing hydrogen from light fractions and/or off-gases from the separator and/or from the various reactors, the integrated fuel plant is made self-sufficient as regards the hydrogen required in the various reactors. At start-up and in case more hydrogen is required than can be provided by the hydrogen reforming/recirculation of the process itself, additional hydrogen from outside the process is used.

For the integrated hydrogen treatment and delivery, the equipment includes lines for feeding off-gases from any one of the reactors selected from the hydrodeoxygenation reactor, the cracking/isomerization reactor, the Fischer-Tropsch reactor and the hydroisomerization reactor to the hydrogen separation unit and lines for feeding recovered and/or reformed hydrogen to any one of said reactors.

In the doubly integrated fuel/wood-processing plant the equipment further comprises means for feeding water produced in the fuel process reactor(s) to the waste water treatment unit of the pulp and paper mill. The equipment also includes means for feeding energy produced in the Fischer-Tropsch reactor to a drying process of said pulp and paper mill. Means are also provided for feeding biomass such as wood-based waste of the pulp and paper mill to the synthesis gas production for the Fischer-Tropsch reactor. Similarly there are means for feeding tall oil/tall oil fatty acids from the pulp and paper mill to the hydrodeoxygenation reactor.

The invention will now be illustrated with examples and with reference to the drawings.

### EXAMPLE 1

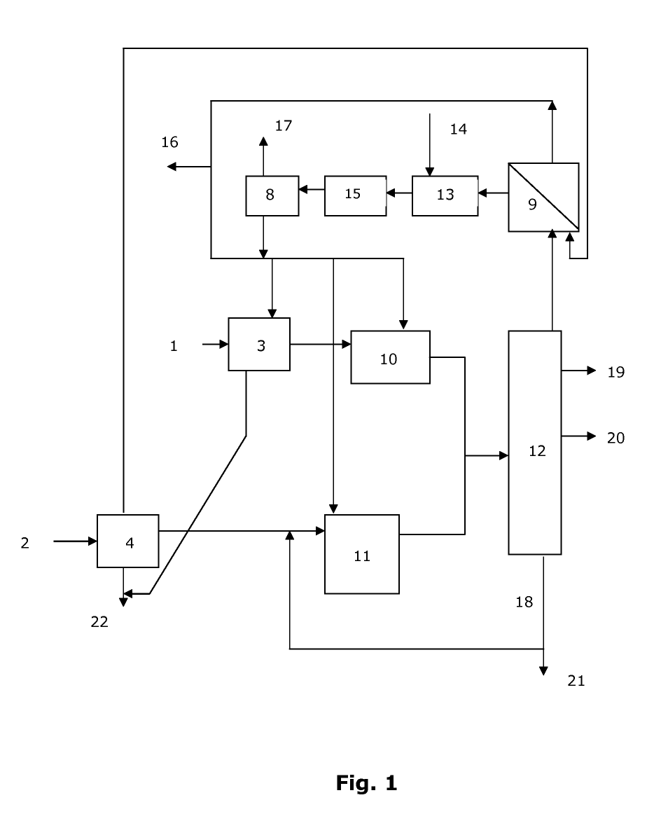

FIG. 1 shows a schematic flow diagram of an integrated process operating according to the invention. The integrated process facilities are located in close connection with a Kraft pulp mill. The two main processes of the invention comprise production of the HDO hydrocarbons by the unit operations 1, 3, 10 and the FT hydrocarbons by the unit operations 2, 4, 11. The hydrocarbons are combined in separation unit 12 and a desired product fraction is recovered at 20.

A feed of biological hydrocarbons comprising crude tall oil from the Kraft mill is optionally thermally refined at 1 and is passed to catalytic HDO reactor 3 charged with a standard hydrodeoxygenation/desulphurization catalyst based on CoMo/Al₂O₃. In an alternative embodiment, a NiMo/Al₂O₃ catalyst is used. The saturated normal paraffins from the HDO reactor 3 have a chain length within the desired diesel or middle distillate range of C₁₁ to C₂₀ and, in the case of tall oil, the fatty acids are predominantly C₁₅ to C₁₈ and, in fact, the chain length is almost purely C₁₈, which provides a diesel fuel with a very high cetane number. However, the cloud point of the n-paraffinic HDO hydrocarbons tends to be too high for arctic use. In order to lower the cloud point, the n-paraffin stream is directed to a further hydroisomerization reactor 10.

Because, the product from HDO step 3 has an ideal chain length, a non-cracking catalyst is used in the hydroisomerization 10. The catalyst is Pt/SAPO-11, which is known to possess a low cracking activity. It should be noted that an excessive cracking character of the isomerization catalyst would be detrimental to the end product because of cutting down the diesel yield.

Paralleling the HDO process, a feed of purified (tar and sulphur free) synthesis gas is produced at 2 by gasification of wood bark and wood slash, which is a waste product of the Kraft mill. The resulting synthesis gas having a H₂/CO-volume ratio of about 2 is fed to FT reactor 4. The pressurized FT reactor 4 is an ARGE type fixed bed reactor, although any other FT reactor could be used and operated e.g. according to the three-phase slurry process of the SASOL Synthol process (see e.g. U.S. Pat. No. 4,906,671). The reactor is charged with a commercially available Co on alumina Fischer-Tropsch catalyst.

The hydrocarbons created in the FT reactor comprise C₁ to very long (C₁₀₀₊) hydrocarbons. After removal of the off-gases consisting of unconverted synthesis gas, methane and light hydrocarbons up to C₄, a wax stream comprising a mixture of paraffinic hydrocarbons from C₅ to C₁₀₀₊ is obtained, which is solid at ambient temperature. The FT wax is not as such a suitable component for liquid fuels and needs to be cracked to chain lengths more suitable for liquid fuels. This is performed in cracking/isomerization reactor 11, which is charged with a Pt/ZSM-23 catalyst known to have a considerable cracking-hydroisomerization activity.

Both the HDO and FT reactions produce water as an undesired by-product in addition to the hydrocarbon streams. The water streams are merged and purified in the existing waste water facility (not shown) of the closely located Kraft mill.

Hydrodeoxygenation reactor 3, as well as the two isomerization reactors 10 and 11, operate under hydrogen pressure. The Fischer-Tropsch reactor 4 also uses hydrogen for its synthesis. Thus, it is evident that several unit processes of the present invention need hydrogen as a reactant or are dependent on hydrogen as the reaction media. In order to provide hydrogen to the unit processes and to recover any unused hydrogen the integrated process provides an extensive recovery, recirculation and reforming of hydrogen.

Light off-gases (CO, H₂, CO₂, Cₗ to C₄) of the top fraction of the separation unit 12 as well as light fractions form the process units 3 (not shown) and 4 are collected and fed via appropriate lines to a hydrogen permeable membrane unit 9 for hydrogen recovery. In an alternative operation mode (not shown in FIG. 1), off-gases from the isomerization stages 10 and 11 are also fed via lines to the membrane 9. The non-permeable mixture of hydrocarbons and other gases from membrane unit 9 is directed to an autothermal reformer 13 to be processed to a mixture of H₂, CO, CO₂, N₂, and H₂O. The product gas mixture from reformer 13, acts as a supply for the subsequent water gas shift (WGS) reactor 15, which produces more hydrogen by the reaction between carbon monoxide and water. Hydrogen is separated in the following pressure swing adsorption (PSA) unit 8 and is fed into a hydrogen recirculation line for feeding the various processes. Extra hydrogen, when available, is fed into the synthesis gas pool 16 to align the H₂/CO-ratio towards the desired. All remaining gases, mainly CO₂ and N₂, are vented through the line 17.

In the case that the in situ produced hydrogen is not adequate for the total hydrogen consumption, additional hydrogen is introduced from an outside source or produced by feeding the reformer 13 with methanol 14 from the outside.

The complete outlets, both gas and liquid phases of hydroisomerization 10 and cracking/hydroisomerization 11 in the proportion of 40% to 60% FT and 60% to 40% HDO hydrocarbons are directed to the separation unit 12, where un-reacted gases and any gaseous hydrocarbons (C₁ to C₄) formed by cracking are separated as a top fraction and directed to membrane separation 9 and to the subsequent reforming. Separation unit 12 distills the mixed hydrocarbon stream and recovers fractions according to selected boiling point ranges. A naphtha fraction consisting essentially of C₅ to C₁₀ hydrocarbons boiling below 150° C. is collected at 19 and a middle distillate fraction consisting essentially of C₁₁ to C₂₀ hydrocarbons boiling between 150° C. and 350° C. is recovered at 20 as a diesel fuel.

The diesel fraction is enriched in C₁₅ to C₁₈ hydrocarbons and especially in C₁₈ hydrocarbons and has an excellent cetane number 60 to 70 and a low cloud point.

Most or about half of the bottom fraction 18 consisting of the heaviest part of the hydrocarbons (>C₂₁) is recirculated to the cracking/hydroisomerization 11 to be cracked and hydroisomerized to diesel range C₁₁ to C₂₀ hydrocarbons having the cloud point targeted. The other part of the bottom fraction, if any, is directed to vacuum distillation 21 for separating a fraction between boiling points 350° C. to 490° C. to be refined to lubricant base oils.

### EXAMPLE 2

An integrated process using tall oil fatty acid (TOFA) as HDO feed and synthesis gas from wood slash, waste wood and bark as FT feed is operated in the process facilities described in Example 1. The two hydrocarbon streams (I.) and (E.) are mixed and distilled to provide a middle distillate (M.), a naphtha fraction (N.), a top fraction (O.) and a bottom fraction (S.), The top fraction is directed to the hydrogen recovery process and the bottom fraction is recirculated to the cracking/isomerization 11.

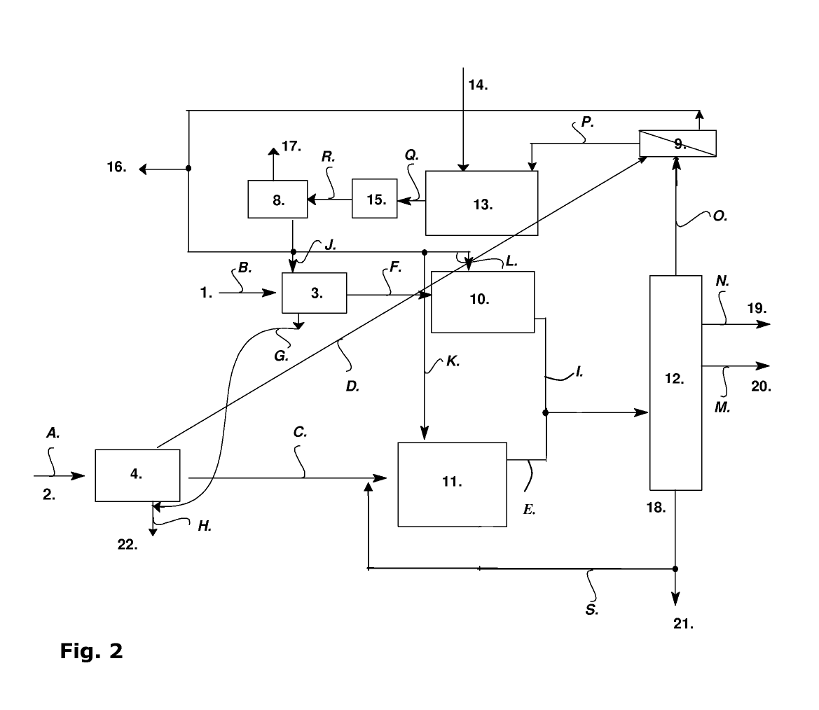

FIG. 2 shows the measurement points for the mass balances in the flow chart of FIG. 1. The amounts of feeds and product as well as the mass balances along the measurement points A. to S. are indicated in Table 1 below. The unit kt/a indicates kilo tons (1000 metric tons) per year.

The mass balance shows that the desired product fraction of diesel fuel (M.) is the largest product fraction. Due to the high level of C₁₈ hydrocarbons in the TOFA feed to the HDO reactor, the diesel fuel fraction has a very high proportion of C₁₈ and a consequent high cetane value.

TABLE 1

| | Point of measure | | | | | | | | | |
|---|---|---|---|---|---|---|---|---|---|---|
| | A. | B. | C. | D. | E. | F. | G. | H. | I. | J. |
| H₂ w % | | | | 3.4 w % | | | | | | 2.8 kt/a |
| CO | | | | 46.7 w % | | | | | | |
| H₂O | | | | | | | 12.8 kt/a | 362.9 kt/a | | |
| TOFA | 100 kt/a | | | | | | | | | |
| Synthesis gas | | 22.6 kg/s | | | | | | | | |
| N₂ | | | | | | | | | | |
| C₁-C₄ | | | | 49.9 w % | 0.4 w % | | | | 2.3 w % | |
| C₅-C₁₀ | | | 27.7 w % | | 25.5 w % | | | | 8.7 w % | |
| C₁₁-C₂₀ | | | 41.9 w % | | 48.5 w % | 8.9 kt/a | | | 89.4 w % | |
| C₂₁-Cₓₓ | | | 30.4 w % | | 25.6 w % | | | | | |
| CO₂ | | | | | | | | | | |

| | Point of measure | | | | | | | | |
|---|---|---|---|---|---|---|---|---|---|
| | K. | L. | M. | N. | O. | P. | Q. | R. | S. |
| H₂ w % | 0.43 kt/a | 0.3 kt/a | | | | | 1.0 kt/a | 4.0 kt/a | |
| CO | | | | | | 33.4 kt/a | 75.3 kt/a | | |
| H₂O | | | | | | | 4.8 kt/a | 40.4 kt/a | |
| TOFA | | | | | | | | | |
| Synthesis gas | | | | | | | | | |
| N₂ | | | | | | | 38.7 kt/a | | |
| C₁-C₄ | | | | | 2.9 kt/a | 38.6 kt/a | | | |
| C₅-C₁₀ | | | | 63.0 kt/a | | | | | |
| C₁₁-C₂₀ | | | 185.1 kt/a | | | | | | |
| C₂₁-Cₓₓ | | | | | | | | | 55.4 kt/a |
| CO₂ | | | | | | | 1.0 kt/a | 116.0 kt/a | |

### EXAMPLE 3

The fuel production equipment according to Example 1 is integrated with a pulp and paper mill. Waste biomass from the pulp mill is used to produce synthesis gas for the Fischer-Tropsch reaction. Tall oil fatty acid (TOFA) from the pulp mill tall oil production facilities is used directly in the HDO reactor to produce n-paraffins. Waste water from the fuel process is directed to the water treatment unit of the pulp and paper mill. Hydrogen sulfide from the synthesis gas production and from the HDO is fed directly into the sulfur recovery and recirculation system of the pulp mill. Energy produced in the exothermic FT reaction is fed to the paper mill and used for drying of paper.

Three different ratios of TOFA and synthesis gas are used as indicated in Table 2. A middle distillate boiling at 175° C. to 350° C. is recovered as a diesel fuel fraction in the separator 12. The top fraction comprises C₁-C₄ hydrocarbons and other gases including hydrogen. This light fraction is sent to the hydrogen separation unit 9. A naphtha fraction containing C₅ to C₁₀ hydrocarbons is recovered above the middle distillate, while the heavy bottom fraction containing C₂₁ to C₁₀₀₊ hydrocarbons is recirculated to the cracking/isomerization reactor 11.

The results are indicated in Table 2, wherein kt/a indicates 1000 metric tons (kilo tons) annually for a process which is run for 8000 hours per annum.

TABLE 2
Feed mixtures and product fractions

| | Feed mixture | | | | | |
|---|---|---|---|---|---|---|
| | 20 kt/a TOFA | | 100 kt/a TOFA | | 100 kt/a TOFA | |
| Product | and 651 kt/a | | and 651 kt/a | | and 130 kt/a | |
| distribution | synthesis gas | | synthesis gas | | synthesis gas | |
| C₁-C₄ | 1.3 | kt/a | 2.9 | kt/a | 2.2 | kt/a |
| C₅-C₁₀ | 56.8 | kt/a | 63.0 | kt/a | 18.8 | kt/a |
| C₁₁-C₂₀ | 121.0 | kt/a | 185.1 | kt/a | 101.1 | kt/a |
| C₂₁-C₁₀₀₊ | 55.4 | kt/a | 55.4 | kt/a | 11.1 | kt/a |
| C₁₆-C₁₈ (FT) | 16.0 | kt/a | 16.0 | kt/a | 3.7 | kt/a |
| C₁₆-C₁₈(TOFA) | 19.3 | kt/a | 80.0 | kt/a | 80.0 | kt/a |

The results indicate that the middle distillate fraction (C11-C20) is enriched in C₁₆ to C₁₈ hydrocarbons from the TOFA feed in all three ratios.

The present invention has been described herein with reference to specific embodiments. It is, however clear to those skilled in the art that the process(es) may be varied within the bounds of the claims.

1. An integrated process for producing diesel fuel from biological material, characterized by the steps of
a. providing a first hydrocarbon stream comprising C₅ to C₁₀₀₊ hydrocarbons by catalytic cracking/isomerization of Fischer-Tropsch paraffins of biological origin to increase the proportion of diesel hydrocarbon C₁₁ to C₂₀ paraffins,
b. providing a second hydrocarbon stream comprising predominantly C₁₅ to C₁₈ hydrocarbons by catalytic hydrodeoxygenation of biological hydrocarbons,
c. mixing said first and second hydrocarbon streams,
d. fractionating the resulting mixed hydrocarbon stream, and
e. recovering a middle distillate fraction.

2. The process according to claim 1, wherein said first hydrocarbon stream is provided by gasification of biomass feedstock to provide synthesis gas; Fischer-Tropsch reaction of said synthesis gas to provide C₁ to C₁₀₀₊ paraffins; and cracking/isomerization of the resulting C₅ to C₁₀₀₊ paraffins to increase the proportion of diesel hydrocarbon C₁₁ to C₂₀ paraffins in the resulting first hydrocarbon stream.

3. The process according to claim 1, wherein said second hydrocarbon stream is provided by hydrodeoxygenation of said biological hydrocarbon feedstock to provide a stream of saturated predominantly C₁₅ to C₁₈ n-paraffins; and non-cracking hydroisomerization of said n-paraffins to increase the proportion of i-paraffins in the resulting second hydrocarbon stream.

4. The process according to claim 1, wherein a top fraction boiling at a temperature lower than said middle distillate fraction is used in said integrated process for providing hydrogen for the catalytic reaction(s) which provide(s) said first and/or said second hydrocarbon stream(s).

5. The process according to claim 1, wherein 5% to 95% of said first and 5% to 95% of said second hydrocarbon streams are combined in a separator, in which the resulting mixed stream is fractionated into a middle distillate comprising C₁₁ to C₂₀ hydrocarbons and a light fraction comprising hydrogen and C₁ to C₄ hydrocarbons, and said middle distillate is recovered for use as diesel fuel.

6. The process according to claim 5, wherein said light fraction is directed to a hydrogen separation unit to separate hydrogen for recirculation to said cracking/isomerization step, to said hydrodeoxygenation step and/or to said hydroisomerization step.

7. The process according to claim 6, wherein a light fraction and/or off-gas of said Fischer-Tropsch paraffins, which contains hydrogen, carbon monoxide and/or C₁ to C₄ hydrocarbons, is also directed to said hydrogen separation unit.

8. The process of claim 7, wherein said hydrogen separation unit comprises a hydrogen permeable membrane and the C₁ to C₄ hydrocarbons and carbon monoxide from said membrane are directed to an autothermal reforming unit to produce a gas comprising hydrogen, carbon monoxide, carbon dioxide, nitrogen and water.

9. The process according to claim 8, wherein said gas or a part thereof is supplied to a water gas shift unit to produce more hydrogen from carbon monoxide and water.

10. The process according to claim 9, wherein said hydrogen is separated in a pressure swing adsorption unit for recirculation in the integrated process to said hydrodeoxygenation step, hydroisomerzation step and/or cracking/isomerization.

11. The process according to claim 8, wherein additional hydrogen required in the process is produced by feeding methanol, preferably biomethanol to said autothermal reforming unit.

12. The process according to claim 5, wherein a heavy fraction comprising C₂₁ to C₁₀₀₊ hydrocarbons and a naphtha fraction comprising C₅ to C₁₀ hydrocarbons is also recovered.

13. The process according to claim 12, wherein at least a part of said heavy fraction is recirculated to said cracking/isomerization to be cracked and hydroisomerized to increase the amount of C₁₁ to C₂₀ hydrocarbons in said first hydrocarbon stream.

14. The process according to claim 1, wherein said fractionation is set to provide a distillate fraction consisting essentially of C₁₅ to C₁₈ hydrocarbons.

15. The process according to claim 1, wherein the biological origin of said first hydrocarbon stream comprises biomass selected from virgin and waste materials of plant, animal and/or fish origin, and wherein said biological hydrocarbons are based on oils, fats and/or waxes of plant, animal and/or fish origin.

16. The process according to claim 15, wherein said biomass is selected from municipal waste, industrial waste or by-products, agricultural waste or by-products, waste or by-products of the wood-processing industry, waste or by-products of the food industry, marine plants and combinations thereof.

17. The process according to claim 1, wherein said process is operated in connection with of a wood-processing industry and the biological feeds to said integrated process originate in or as by-products or waste of said wood-processing industry.

18. The process according to claim 17, wherein the biological origin of said first hydrocarbon stream comprises waste or by-product(s) of the wood-processing industry and said biological hydrocarbons of said second hydrocarbon stream comprise tall oil or tall oil fatty acids.

19. The process according to claim 17, wherein said wood-processing industry comprises a Kraft mill producing crude tall oil and/or tall oil fatty acids.

20. The process according to claim 19, wherein water produced in the integrated fuel process is supplied to said wood-processing industry for purification and wherein sulphur freed in said fuel process is fed to the sulphur circulation of said wood-processing industry.

21. The process according to claim 1, wherein said integrated fuel process is further integrated with a pulp and paper mill and wherein lignocellulosic material is utilized as raw materials for said fuel process and wherein wastes and energy from said fuel process are handled and/or utilized in unit operations of said pulp and paper mill.

22. Use of lignocellulosic material for producing diesel fuel from purely biorenewable sources, characterized by the steps wherein
a. biomass comprising lignocellulosic material is gasified and used to provide a Fischer-Tropsch paraffin stream, which is then cracked under isomerizing conditions,
b. biological hydrocarbons are hydrodeoxygenated to provide a predominantly C₁₅ to C₁₈ n-paraffin stream, which is optionally isomerized under non-cracking conditions,
c. the two streams are combined and fractionated, and a middle distillate fraction is recovered.

23. The use according to claim 22 characterized in that said lignocellulosic material is waste and/or by-products of the wood-processing industry.

24. A method for narrowing the chain length distribution of a Fischer-Tropsch derived diesel fuel, characterized by the steps of
a. combining 5% to 95% of a Fischer-Tropsch derived hydrocarbon stream of C5 to C100+ hydrocarbons with 5% to 95% of a predominantly C₁₅ to C₁₈ hydrocarbon stream obtained by hydrodeoxygention of biological hydrocarbons,
b. fractionating the combined hydrocarbon stream, and recovering a fraction of C₁₁ to C₂₀ hydrocarbons.

25. The method according to claim 24, wherein the recovered fraction contains at least 25% and preferably about 40% to 80% or more of C₁₅ to C₁₈ hydrocarbons.

26. A biological middle distillate fraction obtainable by the processes according to claim 1 from a mixed hydrocarbon stream comprising from 5% to 95% of Fischer-Tropsch derived hydrocarbons having an increased amount of carbon atoms between 11 and 20 and from 5% to 95% of hydrocarbons having predominantly 15 to 18 carbon atoms.

27. The middle distillate fraction according to claim 26, wherein said fraction contains at least 25% and preferably about 40% to 80% or more of C₁₅ to C₁₈ hydrocarbons.

28. Use of the biological middle distillate fraction obtained according to claim 1 as an additive for improving the cetane value of a fuel produced by other means.

29. The use according to claim 28, wherein said fraction is also used for improving the cloud point and/or pour point of said fuel produced by said other means.

30. Equipment for producing fuel from biological material, said equipment comprising
i. a hydrodeoxygenation reactor (3) for hydrodeoxygenating a feed of biological hydrocarbons,
a cracking/isomerization reactor (11) for catalytically cracking and isomerizing a feed of Fischer-Tropsch paraffins of biological origin, and
iii. a separation unit (12) for distilling a combined feed of hydrocarbons provided from said reactors (3, 11) and for the recovery of a middle distillate boiling at 150° C. to 400° C. and the separation of a top fraction boiling at a lower temperature.

31. The equipment according to claim 30, wherein said equipment further comprises
iv. a hydrogen separation unit (9) for separation of hydrogen from said top fraction and means for feeding said hydrogen to said reactors (3, 11), and
v. means (13, 15, 8) for providing of additional hydrogen for said reactors (3, 11) from the non-hydrogen portion of said top fraction.

32. The equipment according to claim 30, wherein said equipment further comprises a hydroisomerization reactor (10) downstream of said hydrodeoxygenation reactor (3) and a Fischer-Tropsch reactor (4) upstream of said cracking/isomerization reactor (11).

33. The equipment according to claim 31, wherein said means for providing additional hydrogen include at least one unit selected form an autothermal reformer (13), a water gas shift reactor (15) and a pressure swing adsorption unit (8).

34. The equipment according to claim 32, which comprises lines for feeding off-gases from any one of the reactors selected from said hydrodeoxygenation reactor (3), said cracking/isomerization reactor, said Fischer-Tropsch reactor (4) and said hydroisomerization reactor (10) to said hydrogen separation unit (9) and lines for feeding recovered and/or reformed hydrogen to any one of said reactors (3, 4, 10, 11).

35. The equipment according to claim 30, which comprises a) means for feeding water produced in said reactor(s) (3, 4) to a waste water treatment unit (22) of a pulp and paper mill and/or b) means for feeding energy produced in said Fischer-Tropsch reactor (4) to a drying process of said pulp and paper mill.

36. The equipment according to claim 34, which comprises a) means for feeding wood-based waste from said pulp and paper mill to a reactor for producing synthetic gas for said Fischer-Tropsch reactor (4) and/or b) means for feeding tall oil and/or tall oil fatty acid from said pulp and paper mill to said hydrodeoxygenation reactor (3).
